(19) United States
(12) Patent Application Publication
(10) Pub. No.: US 2021/0196838 A1
(43) Pub. Date: Jul. 1, 2021

(54) COMPOUNDS AND COMPOSITIONS FOR POTENTIATION OF TLR AGONISTS

(71) Applicant: MIREXUS BIOTECHNOLOGIES INC., Guelph (CA)

(72) Inventors: Jondavid DE JONG, Guelph (CA); Stephanie DEWITTE-ORR, Waterloo (CA); Karl Michael KLINGER, Guelph (CA); Emily MOORE, Guelph (CA); Sheetij GHOSHAL, Mississauga (CA); Anton KORENEVSKI, Guelph (CA)

(21) Appl. No.: 17/271,071

(22) PCT Filed: Aug. 23, 2019
(86) PCT No.: PCT/CA2019/051167

§ 371 Date: Feb. 24, 2021

Related U.S. Application Data
(60) Provisional application No. 62/722,687, filed on Aug. 24, 2018.

(51) Int. Cl. A61K 47/69; A61K 45/06; A61P 31/14; A61K 39/39; A61K 9/51

(52) U.S. Cl. CPC A61K 47/6939; A61K 45/06; A61K 2039/55561; A61K 39/39; A61K 9/5161; A61P 31/14

(57) ABSTRACT

The present invention relates to novel Toll-like Receptor (TLR) agonist compositions that can demonstrate enhanced innate immune responses, improved stability and lower toxicity than said agonists alone. These immunomodulators comprise one or more TLR agonists linked to glycogen-based nanoparticles. Also provided are processes for manufacturing, and methods of using the compositions to induce therapeutic immune responses.

This application claims priority from U.S. Application No. 62/722,687 filed Aug. 24, 2018 which is incorporated herein by reference.

### FIELD OF THE INVENTION

The present invention relates to immune-therapeutic compositions and, in particular, immune-therapeutic compositions, comprising Toll-like Receptor (TLR) agonists.

### BACKGROUND OF THE INVENTION

An immune response is a complex process involving many interacting components within both the innate and adaptive arms of the immune system. Proper activation of the innate immune system is not only required to control infection and cancer but is also imperative for the initiation and engagement of the adaptive immune response. Mechanistically, the magnitude and characteristics of innate immune responses are, in part, driven by engagement of a highly conserved family of pattern recognition receptor proteins termed Toll-like Receptors (TLRs). One of the primary roles of TLRs is to recognize pathogen-associated molecular patterns (PAMPs) and danger-associated molecular patterns (DAMPs); engagement of TLRs by their agonists results in rapid induction of the innate immune response.

There are 10 defined TLRs in humans, aptly named TLR 1-10. TLRs are widely expressed by many cell types. These include subsets of immune cells such as Dendritic Cells (DCs)-professional antigen-presenting cells that play an important role in the induction and maintenance of innate and adaptive immune responses. TLRs are also expressed on non-immune cells such as epithelial cells and fibroblasts. TLRs 1, 2, 4, 5, 6, and 10 are expressed on the cell surface, whereas the nucleic acid sensing TLRs 3, 7, 8, and 9, are sequestered in intracellular compartments (endosomes). Each TLR has specific agonists which activate immune signalling; the cell surface expressed TLRs tend to respond to bacterial cell components such as peptidoglycans (TLRs 1 and 2), lipoproteins (TLR 1 and 6), lipopolysaccharides (TLR 4), flagella (TLR 5) and di/tri acylated lipopeptides (TLR 10). The endosomal TLR respond to viral and bacterial nucleic acid-based agonists such as double-stranded RNA (TLR 3), single-stranded RNA (TLR 7 and 8), and CpG DNA (TLR 9).

Given TLRs' ability to modulate the immune response, there is a great deal of interest in compositions and formulations engaging TLRs for the treatment and prevention of infection (viral, bacterial, parasitic), cancer, and immunodeficiencies. Indeed, there are several reports that indicate proper engagement of TLRs by their cognate agonists can protect against bacterial and viral infection as well as induce anti-tumour immune responses in laboratory animals. Toxic side effects have been observed in lab animals including fever, changes in liver function, hematopoiesis, and blood clotting. Clinical trials conducted with TLR agonists have shown some efficacy in the treatment of viral diseases (eg. Hepatitis C, herpes simplex 2), some forms of cancer (e.g. melanoma, squamous cell carcinoma) and autoimmune conditions (eg. asthma, chronic fatigue syndrome). However, in humans, a relatively small therapeutic window has been observed as the efficacy has been limited by low circulation time, poor solubility and systemic toxicity. Toxic manifestations after parenteral administration in humans include transient fever, minor liver enzyme change and a slight decrease in circulating leukocyte numbers. As a result, there are only three TLR agonists that are currently FDA approved for use in humans—(i) imiquimod (TLR7 agonist) for topical treatment of superficial basal cell carcinoma, actinic keratosis and genital warts; (ii) monophosphoryl lipid A (MPL) as part of GlaxoSmithKline's Cervarix, an intra-muscular vaccine against human papillomavirus (HPV); and (iii) bacillus Calmette-Guerin (BCG) for intravesical immunotherapy of in situ bladder carcinoma. Toxicity-related adverse events in clinical trials of other TLR agonists, especially when delivered intravenously, have prevented further clinical development. Intravenous delivery of TLR agonists also faces the challenge of rapid metabolism by serum enzymes which results in a short drug-half life. Modified versions of TLR agonists with longer circulation times have been created (see, e.g., U.S. Pat. No. 5,532,130) but these tend to prolong the toxic side-effects, which are mostly attributed to nonspecific distribution that results in immune activation in healthy tissue while delivering sub-therapeutic concentrations of the drug to the intended target tissue. Other formulations that incorporate TLR agonists into lipid nanoparticles (see, e.g. U.S. Pat. No. 8,357,374B2) and synthetic polymeric devices (eg. see patent application WO2013106852A1) have been created to improve tissue distribution. However, inherent toxicity (lipid nanoparticles, liposomes) and poor scalability (synthetic nanoparticles) of these nanoparticle systems have limited further clinical development. Conjugation strategies that employ TLR agonist formulations containing targeting molecules (e.g. homing peptides) have also failed to progress into clinical trials. Therefore, there exists a need for novel formulations of TLR agonists that can widen the therapeutic window by producing a therapeutic effect at lower doses of the TLR agonist and mitigate toxic side effects by improving tissue distribution.

### SUMMARY OF INVENTION

The compositions described herein allow for enhanced activation of TLRs, and may further provide improved stability and lower toxicity of the TLR agonists. The glycogen-based nanoparticles can be naturally-derived, or chemically synthesized, unmodified, or chemically modified to carry altered physiochemical characteristics (positive or negative charge, hydrophobicity, or combination thereof). Provided herein are novel compositions of TLR agonists that allow for enhanced activation of TLRs while improving stability and lowering toxicity of the TLR agonists. Also described are methods of using these compositions.

In one embodiment, the formulations comprise a carbohydrate-based nanoparticle and one or more TLR agonists. In one embodiment the carbohydrate-based nanoparticles are glycogen-based nanoparticles. The glycogen-based nanoparticles may be naturally derived, or chemically synthesized, unmodified, or chemically modified to carry altered physiochemical characteristics (positive or negative charge, hydrophobicity, or combination thereof) or targeting moieties. The TLR agonist may be an agonist of TLR1, TLR2, TLR3, TLR4, TLR5, TLR6, TLR7, TLR8, TLR9, TLR10 or any combination thereof. The TLR agonist may be a small molecule or a macromolecule, for instance a lipopolysaccharide or a nucleic acid. In one embodiment the TLR agonist is directly covalently linked to the glycogen nanoparticle or covalently linked via a linker, for instance an amino group or a carboxy group. In one embodiment the TLR agonist is non-covalently linked to the glycogen nanoparticle, for instance via electrostatic interactions, hydrophobic interactions, Van der Waals forces or other non-covalent interactions. In one embodiment the ratio of TLR agonist covalently or non-covalently linked to the glycogen nanoparticle may be tunable.

In one aspect, there is provided an immunomodulator for enhancing the innate immune response comprising: a TLR agonist covalently or non-covalently linked to glycogen-based polysaccharide nanoparticles having a molecular weight of 10⁶ to 10⁷ Da comprising α-D glucose chains, having an average chain length of 11-12, with 1→4 linkage and branching point occurring at 1→6 and with a branching degree of between 6% and 13%.

The TLR agonist may be covalently linked to the nanoparticles through a linking group. In one embodiment, the nanoparticles are cationic and the TLR agonist is non-covalently linked to the nanoparticles through electrostatic interactions.

In some embodiments, the TLR agonist is selected from: double-stranded RNA, double-stranded DNA and single-stranded RNA, single-stranded DNA, or any synthetic analogs thereof including poly IC, CpG ODN, LNA.

In one embodiment, the TLR receptor is in an endosome. In one embodiment, the TLR receptor is located at the cell surface.

The TLR agonist may comprise between about 60% and 600% by weight relative to the polysaccharide nanoparticles.

The nanoparticles suitably have a polydispersity index (PDI) of less than about 0.3 as measured by dynamic light scattering and an average particle diameter of between about 10 nm and 150 nm before being covalently or non-covalently linked to the TLR agonist.

The nanoparticles may be further covalently linked to one or more small molecules for directing the nanoparticles to a type of cell or cellular compartment.

Also provided is a pharmaceutical composition comprising the immunomodulator as described herein and a pharmaceutically acceptable excipient.

The pharmaceutical composition may be a powder, tablet or capsule.

The pharmaceutical composition can further include an antiviral agent, an anticancer agent, a further immunomodulator, or a vaccine.

In one embodiment, the pharmaceutical composition is a vaccine.

In another aspect, there is provided a method of stimulating an innate immune response in a subject comprising administering to the subject a therapeutically effective amount of an immunomodulator or a pharmaceutical composition as described herein.

The immunomodulator or pharmaceutical composition may be administered by intramuscular, oral, sublingual, buccal, parenteral, nasal, subcutaneous, intradermal, or topical administration.

In one embodiment, the method is for the prevention or treatment of a viral infection in the subject.

In one embodiment, the method is for cancer immunotherapy.

In another aspect, there is provided a method of potentiating a TLR agonist comprising covalently or non-covalently linking the TLR agonist to glycogen-based polysaccharide nanoparticles having a molecular weight of 10⁶ to 10⁷ Da comprising α-D glucose chains having an average chain length of 11-12, with 1→4 linkage and branching point occurring at 1→6 and with a branching degree of between 6% and 13%. In one embodiment, the nanoparticles are cationized. In some embodiments, the TLR agonist is selected from: double-stranded RNA, double-stranded DNA and single-stranded RNA, single-stranded DNA, or any synthetic analogs thereof including poly IC, CpG ODN, LNA.

An aspect of the present invention relates to the use of the novel formulations of TLR agonists to induce an anti-viral response in a subject. Surprisingly, when tested in cell culture, the novel formulations provided in the invention induced a robust anti-viral response in cells at concentrations where the TLR agonist alone was ineffective.

Another aspect of the present invention relates to the use of the novel formulations of the invention to activate immune cells (dendritic cells, macrophages, natural killer cells, natural killer T-cells, T-cells, tumour-infiltrating leukocytes). A further aspect of the invention relates to the use of the novel formulations of the invention for immunotherapy of cancer, as a monotherapy or in combination with other therapies (e.g. radiation,).

The novel formulations of the invention can be in the form of an aerosol, dispersion, solution, or suspension. The formulations can be used for intramuscular, oral, sublingual, buccal, parenteral, nasal, subcutaneous, intradermal, or topical administration. The present invention is also directed to methods of immunizing a subject or treating or preventing various diseases or disorders in the subject by administering to the subject an effective amount of the immunological formulations.

### BRIEF DESCRIPTION OF FIGURES

FIG. 1 shows levels of IFN1 expression at the transcript level following treatment with poly IC or poly IC:PhG. RTgutGC cells were treated with media alone (CTRL), 1 μg/mL of poly IC and 1 μg/mL of poly IC covalently linked to 6.875 μg/mL PhG (poly IC:PhG) for 24 hours. Interferon 1 (IFN1) transcript levels were measured using qRT-PCR.

FIG. 2 shows levels of vig3 expression at the transcript level following treatment with poly IC or poly IC:PhG. RTgutGC cells were treated with media alone (CTRL), 1 μg/mL of poly IC and 1 μg/mL of poly IC covalently linked to 6.875 μg/mL PhG (poly IC:PhG) for 24 hours. Vig3 transcript levels were measured using qRT-PCR.

FIG. 3 shows the ability of cationized phytoglycogen nanoparticles (Cat-PhG) to bind poly IC as demonstrated by electrophoretic mobility shift assay (EMSA). Three (3) micrograms of poly:IC was mixed with 2-fold dilution series of Cat-PhG harbouring a 0.88 degree of substitution (ds). After a 20-minute incubation at room temperature, samples were separated on a 1% agarose gel, stained with ethidium bromide, and subsequently imaged under UV light. Shown are the EMSA gels for Cat-PhG-0.88 demonstrating maximum loading ˜6:1 (poly:IC:PhG w/w).

FIG. 4 shows PhG bound TLR3 agonist induces greater expression of interferon stimulated genes than “free” agonist. Per Example 17, quantitative reverse-transcriptase polymerase chain reaction (qRT-PCR) reactions were used to quantify the amount of ISG transcript in mock, “free” poly:IC treated, or PhG-poly:IC treated Human Embryonic Lung (HEL-299) and Human Foreskin Fibrobalst (HFF-1) cells.

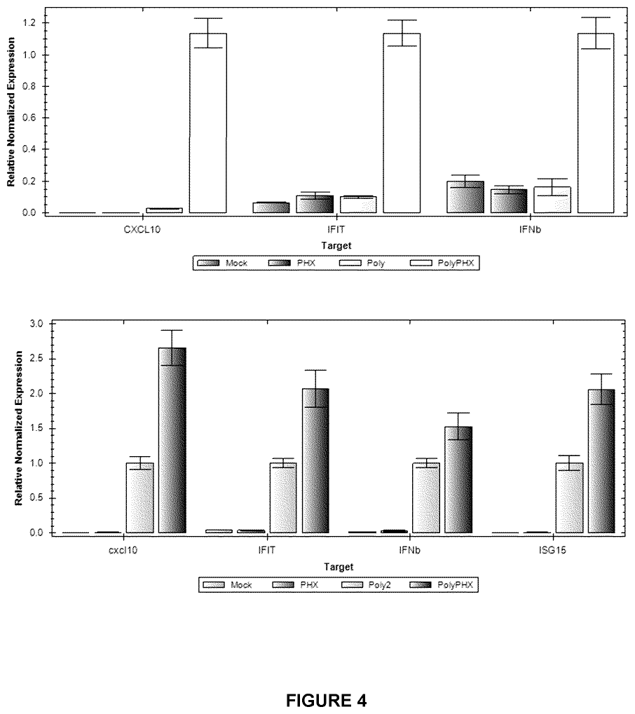

FIG. 5 shows PhG bound TLR3 agonist provides greater protection from viral infection than “free” agonist. Per Example 18, HEL-299 cells were either mock treated, treated with 400 ng/ml “free” poly:IC, or treated with 400 ng/ml poly:IC coupled to Cat-PhG at a 0.4:1 ratio (polylC:PhG, w/w). After 6-hours of treatment, cells were infected with VSV-GFP at a MOI of 0.1 and cells were visualized the next day under fluorescent and brightfield microscopy.

FIG. 6 shows PhG bound TLR3 agonist provides greater protection from viral infection than “free” agonist over a broad range of concentrations at 6-hours pre-treatment. Per Example 18, HEL-299 and HFF-1 cells were treated with a 2-fold dilution series of “free” poly:IC or poly:IC coupled to PhG at a 1:1 ratio (w/w). After 6-hours of treatment cells were infected with VSV-GFP at a MOI of 0.1 and percent infection was determined by measuring GFP fluorescence via plate reader.

FIG. 7 shows dose-response curve of PhG bound TLR3 agonist and “free” agonist. PhG bound TLR3 agonist provides greater protection from viral infection than “free” agonist at 1-hour pre-treatment. Data from FIG. 6 was plotted in a normalized, transformed dose vs. response curve to accurately calculate EC50 values. PhG bound TLR3 agonist demonstrated a 21-fold decrease in EC50 compared to free agonist (p<0.0001).

FIG. 8 shows PhG bound TLR3 agonist provides greater protection from viral infection than “free” agonist over a broad range of concentrations at 1-hour pre-treatment. Per Example 18, HEL-299 cells were treated with a 2-fold dilution series of “free” poly:IC, or poly:IC coupled to PhG at a 1:1 ratio (w/w). After 1-hours of treatment cells were infected with VSV-GFP at an MOI of 0.1. Percent infection was determined by measuring GFP fluorescence via plate reader.

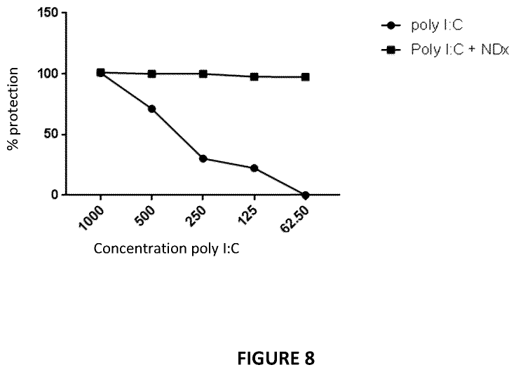

FIG. 9 shows PhG enhances uptake and intracellular stability of TLR 3 agonist in rainbow trout gut cells (RT-gut). Per Example 19, low molecular weight poly:IC was labelled with a fluorescent dye, then left as “free” poly:IC or coupled with cationic PhG (0.38) at a 1:1 (w/w) ratio. Free poly:IC or PhG coupled poly:IC was added to RT-gut cells at a concentration of 1 ug/ml and incubated at 4° C. or 20° C. for 4 hours. Cells were imaged via fluorescent microscopy, and average intracellular fluorescence intensity was measured from 5 randomly selected cells.

FIG. 10 shows PhG enhances uptake and intracellular stability of TLR 9 agonist in Chicken macrophage cell line Hd-11 cells. Per Example 20, HD-11 Cells were dosed in triplicate with different doses of PhG-CpG formulations and incubated at 37° C. for 2 hours in basic media. After removing the cell supernatants from the treated cells, the cell pellets were re-suspended in PBS mixed with MitoTracker™ Green FM (Life Technologies) cell viability stain for flow cytometry. The CpG-ODN uptake and cell viability were assessed using the Attune® Acoustic Focusing Flow Cytometer. CpG uptake was measured as percentage of cells positive for Alexa Fluor 647 CpG-ODN red fluorescence following treatment. The viability was assessed using MitoTracker™ Green FM viability dye. The CpG-ODN uptake was calculated based on the percentage of viable cells that exhibited a fluorescence signal above the threshold signal.

FIG. 11 shows PhG enhances macrophage stimulation by TLR9 agonists at low concentrations. Per Example 21, HD-11 Cells were dosed in triplicate with different doses of PhG-CpG formulations and incubated at 37° C. for 2 hours in basic media. After the 24-hour incubation, the Griess assay (standard Griess Assay Kit, Life Technologies) was used to evaluate nitrite production. The absorbance was read at 548 nm using a microplate reader and nitrite concentration was calculated using a nitrite standard curve (1-100 μM). Statistical analysis was performed using the GraphPad Prism software.

FIG. 12 shows internalization of 1 mg/ml Cy5.5-labelled PhG particles by THP-1 monocytes at various time points at 37 degrees C.

### DETAILED DESCRIPTION

As used herein “immunotherapy” refers to treating or preventing disease by inducing, enhancing or suppressing an immune response. An immunotherapy designed to elicit or amplify an immune response is referred to as an activation immunotherapy. An immunomodulator is an active agent used in immunotherapy.

As used herein “TLR agonist” refers to any compound, natural or synthetic and analogs thereof, capable of binding to TLRs and inducing a signal transduction. In one embodiment the TLR agonist may be selected from the list in Table 1.

TABLE 1
Toll-like receptors and their known natural and synthetic agonists.

| | | Endogenous | |
|---|---|---|---|
| Receptor | Pathogen associated ligand | ligand | Synthetic ligand |
| TLR 1/2 | Triacylated lipopeptides | Unknown | Pam3Cys |
| | (Bacteria and | | |
| | Mycobacteria) | | |
| TLR2 | Peptidoglycan (gram | Unknown | CFA, MALP2, |
| | positive bacteria); Bacterial | | Pam2Cys, FSL-1, |
| | lipoprotein, lipoteichoic | | Hib-OMPC |
| | acid; LPS; GPI-anchor | | |
| | proteins; Neisserial porins; | | |
| | Hemagglutinin; | | |
| | phospholipomannan; LAM | | |
| TLR 3 | Viral dsRNA | Unknown | Poly I:C; Poly A:U |
| TLR 4 | LPS (gram-negative | Hsp60, | AGP, MPL A, RC- |
| | bacteria); F-protein (RSV); | Hsp70, | 529, MDF2β, CFA |
| | Mannan; | fibronectin | |
| | Glycoinositolphospholipids; | domain | |
| | | A surfactant | |
| | | protein A, | |
| | | hyaluronan; | |
| | viral envelope proteins | HMGB-1 | |
| TLR 5 | Flagellin | Unknown | Flagellin |
| TLR 2/6 | Phenol-soluble modulin; | Unknown | MALP-2, |
| | Diacylated lipopeptides; | | Pam2Cys, FSL-1 |
| | LTA; Zymosan | | |
| TLR 7 | Viral ssRNA | Human | Guanosine |
| | | RNA | analogs; |
| | | | imdazoquinolines |
| | | | (eg. Imiquimod, |
| | | | resiquimod, |
| | | | vesatolimod); |
| | | | Loxoribine |
| TLR 8 | ssRNA from RNA virus | Human | Imidazoquinolines; |
| | | RNA | Loxoribine; |
| | | | ssPolyU; 3M-012 |
| TLR 9 | Viral dsDNA; Hemozoin; | Human | CpG- |
| | Unmethylated CpG DNA | DNA/ | oligonucleotides |
| | | chromatin, | (CpG-ODN) |
| | | LL37-DNA | |
| TLR 10 | Unknown | Unknown | Unknown |

As used herein “subject” refers to an organism being treated, in one embodiment an invertebrate organism, in one embodiment a plant, in one embodiment a vertebrate organism, in one embodiment a mammal, in one embodiment a human patient.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

As used herein “treatment” and grammatical variations thereof refers to administering a composition of the present invention, in one embodiment to induce or enhance an immune response to affect an alteration or improvement of a disease or condition, which may include alleviating one or more symptoms thereof, or prophylactically. The treatment may require administration of multiple doses at regular intervals or prior to onset of the disease or condition to alter the course of the disease or condition.

In one aspect, there is provided novel vehicles for TLR agonists to surface and/or endosomal TLRs.

In one embodiment, there is provided a method for potentiating TLR agonists. As used herein, “potentiating a TLR agonist” or grammatical variations thereof refers to one or more of enhancing the innate immune response induced by a TLR agonist, improving stability or lowering toxicity of a TLR agonist.

In one aspect, there is provided compositions for use in stimulating an innate immune response. As used herein, “stimulating an innate immune response” or grammatical variations thereof refers to upregulating gene(s) associated with an innate immune response, for example (and without being limited thereto) VIG3, IFN, CXCL10, MX1 and IFIT1.

Glycogen and phytoglycogen are composed of molecules of α-D glucose chains having an average chain length of 11-12, with 1→4 linkage and branching point occurring at 1→6 and with a branching degree of about 6% to about 13%. Glycogen and phytoglycogen molecules may be modified as described further below; “glycogen-based polysaccharide” refers to a polysaccharide exhibiting this structure although subject to further modifications.

The yields of most known methods for producing glycogen or phytoglycogen and most commercial sources are highly polydisperse products that include both glycogen or phytoglycogen particles, as well as other products and degradation products of glycogen or phytoglycogen.

As used herein “glycogen nanoparticles” is used to refer to both glycogen and phytoglycogen nanoparticles, however, it will be understood that in a preferred embodiment, phytoglycogen nanoparticles are used. Accordingly, unless specifically and explicitly excluded, it will be understood the embodiments described include nanoparticles manufactured from plant starting materials.

“Glycogen” can include both products derived from natural sources and synthetic products, including synthetic phytoglycogen i.e. glycogen-like products prepared using enzymatic processes on substrates that include plant-derived material e.g. starch.

In one embodiment, monodisperse glycogen nanoparticles are used. In a further preferred embodiment, monodisperse phytoglycogen nanoparticles are used. In one embodiment, the monodisperse phytoglycogen nanoparticles are prepared according to Example 1.

Monodisperse phytoglycogen nanoparticles are also commercially available [PhytoSpherix® Mirexus Biotechnologies, Inc.].

These phytoglycogen nanoparticles are non-toxic, have no known allergenicity, and can be degraded by glycogenolytic enzymes (e.g. amylases and phosphorylases) of the human body. The products of enzymatic degradation are non-toxic molecules of glucose. In one embodiment, glycogen refers to glycogen nanoparticles manufactured according to methods described herein. The described methods enable production of substantially spherical nanoparticles, each of which is a single glycogen molecule.

The nanoparticles as taught herein have a number of properties that make them particularly suitable for use in pharmaceutical compositions.

Glycogen nanoparticles are generally photostable and stable over a wide range of pH, electrolytes, e.g. salt concentrations.

Further, their high molecular weight (10⁶-10⁷ Da) is believed to be associated with longer intravascular retention time.

Glycogen nanoparticles may have particular utility in compositions directed to diabetics based on a slower in vivo rate of digestion as compared to starch.

Further, many existing drugs are rapidly eliminated from the body leading to a need for increased dosages. The compact spherical nature of glycogen nanoparticles is associated with efficient cell uptake, while the highly branched nature of glycogen is associated with slow enzymatic degradation.

Glycogen nanoparticles have properties that address a number of requirements for materials used in pharmaceutical and biomedical applications: predictable biodistribution in different tissues and associated pharmacokinetics; hydrophilicity; biodegradability; and non-toxicity.

United States patent application publication no. United States 20100272639 A1, assigned to the owner of the present application and the disclosure of which is incorporated by reference in its entirety, provides a process for the production of glycogen nanoparticles from bacterial and shell fish biomass. The processes disclosed generally include the steps of mechanical cell disintegration, or by chemical treatment; separation of insoluble cell components by centrifugation; elimination of proteins and nucleic acids from cell lysate by enzymatic treatment followed by dialysis which produces an extract containing crude polysaccharides, lipids, and lipopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and freeze drying to produce a powder of glycogen. Glycogen nanoparticles produced from bacterial biomass were characterized by MWt 5.3-12.7×10⁶ Da, had particle size 35-40 nm in diameter and were monodisperse.

Methods of producing monodisperse compositions of phytoglycogen are described in the International patent application entitled “Phytoglycogen Nanoparticles and Methods of Manufacture Thereof”, published under the international application publication no. WO2014/172786, assigned to the owner of the present application, and the disclosure of which is incorporated by reference in its entirety. In one embodiment, the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. In one embodiment of the method, the phytoglycogen-containing plant material is a cereal or a mixture of cereals, in one embodiment, corn. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermo treatments that degrade the phytoglycogen material. The aqueous composition can further be dried.

In one embodiment, the composition is obtained from sweet corn (Zea mays var. saccharata and Zea mays var. rugosa). In one embodiment, the sweet corn is of standard (su) type or sugary enhanced (se) type. In one embodiment, the composition is obtained from dent stage or milk stage kernels of sweet corn. Unlike glycogen from animal or bacterial sources, use of phytoglycogen eliminates the risk of contamination with prions or endotoxins, which could be associated with these other sources.

The polydispersity index (PDI) of a composition of nanoparticles can be determined by the dynamic light scattering (DLS) technique and, in this embodiment, PDI is determined as the square of the ratio of standard deviation to mean diameter (PDI=(σ/d)². PDI can also be expressed through the distribution of the molecular weight of polymer and, in this embodiment, is defined as the ration of Mw to Mn, where Mw is the weight-average molar mass and Mn is the number-average molar mass (hereafter this PDI measurement is referred to as PDI*). In the first case, a monodisperse material would have a PDI of zero (0.0) and in the second case the PDI* would be 1.0.

In one embodiment, the pharmaceutical composition comprises monodisperse glycogen nanoparticles having a PDI of less than about 0.3, less than about 0.2, less than about 0.15, less than about 0.10, or less than 0.05 as measured by DLS. In one embodiment, the pharmaceutical composition comprises monodisperse glycogen nanoparticles having a PDI* of less than about 1.3, less than about 1.2, less than about 1.15, less than about 1.10, or less than 1.05 as measured by SEC MALS.

In one embodiment, there is provided a pharmaceutical composition that comprises, consists essentially of, or consists of a composition of glycogen nanoparticles covalently or non-covalently linked to a TLR agonist. In one embodiment, the TLR agonist is a macromolecule, for example a nucleic acid, a peptide, a peptidoglycan or a lipopolysaccharide. In one embodiment, the TLR agonist is a small molecule. In one embodiment the TLR agonist may be selected from Table 1. In one embodiment the pharmaceutical composition is suitable for use in immunotherapy and, in particular, in an activation immunotherapy.

As used herein, “covalently linked” refers to a link via covalent bond, whether directly or via a linker.

As used herein, “non-covalently linked” refers to all non-covalent interactions including electrostatic interactions, hydrophobic interactions, Van der Waals forces and combinations thereof.

In one embodiment, the glycogen nanoparticles have an average particle diameter of between about 10 nm and about 150 nm, in one embodiment about 30 nm to about 150 nm, in one embodiment about 60 nm to about 110 nm, and in other embodiments, about 40 nm to about 140 nm, about 50 nm to about 130 nm, about 60 nm to about 120 nm, about 70 nm to about 110 nm, about 80 nm to about 100 nm, about 10 nm to about 30 nm.

The methods of producing phytoglycogen nanoparticles as detailed in Example 1 and in the international patent application entitled “Phytoglycogen Nanoparticles and Methods of Manufacture Thereof”, are amenable to preparation under pharmaceutical grade conditions.

### Chemical Modification of Glycogen Nanoparticles

To impart specific properties to glycogen nanoparticles, they can be chemically modified via numerous methods common for carbohydrate chemistry.

The resulting products are referred to herein as functionalized or modified nanoparticles or derivatives. Functionalization can be carried out on the surface of the nanoparticle, or on both the surface and the interior of the particle, but the structure of the glycogen molecule as a single branched homopolymer is maintained. In one embodiment, the functionalization is carried out on the surface of the nanoparticle. As will be understood by those of skill in the art, chemical modifications should be non-toxic and generally safe for human consumption.

In some embodiments of the present invention, it is advantageous to change the chemical character of glycogen from its hydrophilic, slightly negatively charged native state to be positively charged, or to be partially or highly hydrophobic. J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007 provides certain examples of chemical processing of polysaccharides.

Various derivatives can be produced by chemical modification of hydroxyl groups on glycogen, through one or more functionalization steps. Such functional groups include, but are not limited to, nucleophilic and electrophilic groups, acidic and basic groups, e.g., carboxyl groups, amine groups, thiol groups, and aliphatic hydrocarbon groups such as alkyl, vinyl and allyl groups.

In one embodiment, the functionalized nanoparticles are modified with amino groups, which can be primary, secondary, tertiary, or quaternary amino groups, including quaternary ammonium compounds of varying chain lengths. The short-chain quaternary ammonium compound includes at least one alkyl moiety having from 1 to 27 carbon atoms, unsubstituted or substituted with one or more non-carbon heteroatoms (e.g. N, O, S, or halogen).

In certain embodiments, the nanoparticles described are functionalized via glycidyltrimethylammonium chloride (GTAC) to render an overall positive charge. In certain embodiments, two or more different chemical compounds are used to produce multifunctional derivatives.

To perform direct conjugation reactions between native glycogen nanoparticles under aqueous conditions, water-soluble chemicals with reactive or activated functionalities (e.g. epoxide or anhydride, pH 8-11) are often necessary. To maintain stability of the glycogen nanoparticles, solution pH is preferably slightly basic, optimally between 8 and 9. As the hydroxyl moieties of the native glucose subunits are not sufficiently reactive (i.e. deprotonated) under these conditions, a significant excess of reagent may be required to obtain appreciable functionalization. Although some reactions (e.g. with alkyl halides) are best conducted in organic solvents such as dimethyl sulfoxide (DMSO) or dimethyl formamide (DMF) to improve reagent solubility and homogeneity, derivatization in aqueous environment is preferable, as these reaction conditions are generally mild and impart low to minimal toxicity.

An alternative chemical modification strategy involves activation of glycogen by appending a functionalized linker or conducting a functional group interconversion of the hydroxyl group, to a more chemically active group. This can be performed in aqueous or organic media, offering the advantage of higher chemical selectivity and efficiency. It is possible to isolate the activated glycogen precursors (e.g. aminated, carboxylated) which can then be coupled with a suitable reagent. As detailed in Examples 2-13, the present inventors have synthesized a number of nanoparticle functional derivatives using this method. Nucleophilic and electrophilic groups (such as amino or hydrazide and aldehyde groups, respectively) have been attached to the glycogen nanoparticle backbone.

By way of example, the simplest approach is the introduction of carbonyl groups by selective oxidation of glucose hydroxyl groups at positions of C-2, C-3, C-4 and/or C-6. There is a wide spectrum of redox agents which can be employed, such as persulfate, periodate, bromine, acetic anhydride, Dess-Martin periodinane, TEMPO (2,2,6,6-Tetramethylpiperidin-1-yl)oxyl), etc.

Glycogen nanoparticles functionalized with carboxylate groups are readily reactive towards compounds bearing primary or secondary amine groups. The coupling of these two partners (e.g. through EDC coupling chemistry) results in the formation of amides. This chemistry could also be employed in the reverse direction: reacting amine functionalized glycogen with carboxylate-containing compounds.

For the preparation of primary, secondary or tertiary amino-functionalized nanoparticles, one method of the current invention utilizes the reaction of unmodified glycogen with 2-aminoalkyl halides or hydrogen sulfate. Treatment of the glycogen under basic (pH 9-12) conditions with aminoalkyl substrates results in a nucleophilic substitution reaction, displacing the halide or hydrogen sulfate leaving group. As a result, the glycogen is aminoalkylated (e.g. primary, secondary, or tertiary aminated with an O-alkyl linker). The reaction can be performed at a variety of temperatures (25-90° C.) and aminoalkylating agents of varying chain lengths or leaving groups.

For the quaternary ammonium modification of glycogen, glycogen nanoparticles are reacted with a variety of 3-chloro-2-hydroxypropyltrialkylammonium chloride reagents, which exist in an epoxide-chlorohydrin equilibrium, depending on solution pH. Under the basic conditions of the reaction performed herein (pH 9-12), the quaternary ammonium reagents are in the epoxide form, which react readily by base-catalyzed ring-opening with the glycogen. The resulting products are 3-(trimethylammonio)-2-hydroxyprop-1-yl or 3-(N-alkyl-N,N-dimethylammonio)-2-hydroxyprop-1-yl glycogen, where in the latter case, the alkyl groups are long-chain alkyl groups including lauryl (C12), cocoalkyl, (C8-C18), and stearyl (C12-C27).

The above quaternary ammonium modified glycogen can then be further reacted with various alkyl, benzyl, or silyl halides to afford nanoparticles bearing both hydrophilic (cationic, quaternary ammonium) and hydrophobic functionalities.

Another route to primary amination of glycogen includes a two-step sequence involving imides (Examples 7 and 8). Glycogen nanoparticles are first reacted under basic conditions with an imide-containing epoxide or alkyl halide, by the chemistry described above, to provide the corresponding (N-imidyl) protected aminoalkyl glycogen

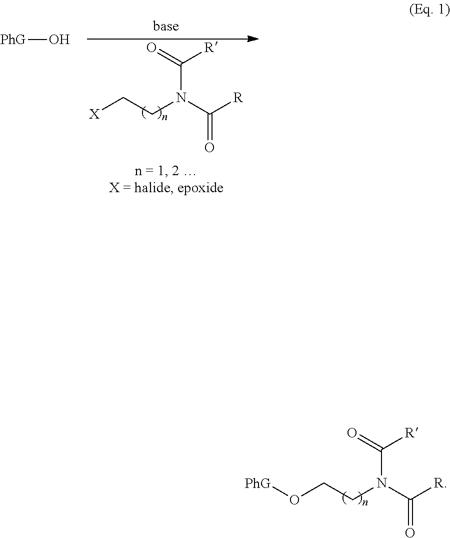

Depending on the nature of the imide reagent used, the length of the O-alkyl tether and substituents (e.g. imide bearing various alkyl/aryl cyclic or acyclic groups) may be tailored. The N-imidyl group on this product can then be removed by one of several conditions (reducing agent followed by acetic acid at pH 5, aqueous hydrazine hydrate, methylamine, etc.) to afford primary aminoalkylated glycogen nanoparticles (Eq. 2).

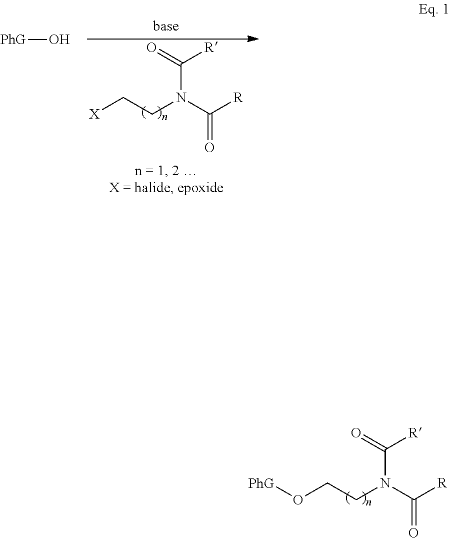

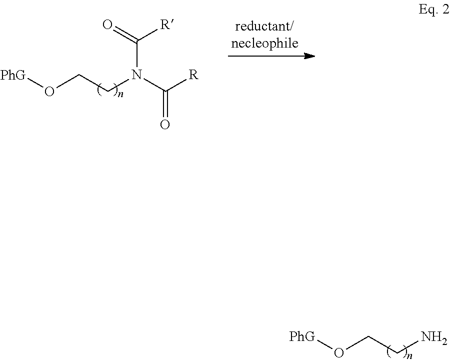

Reductive amination of the nanoparticles can be also achieved by the following two step process. First step is cyanoalkylation, i.e., converting hydroxyls into O-cyanoalkyl groups by reaction with bromoacetonitrile or acrylonitrile. In the second step, the cyano groups are reduced with metal hydrides (borane-THF complex, LiAlH₄, etc).

Amino-functionalized nanoparticles are amenable to further modifications. Amino groups are reactive to carbonyl compounds (aldehydes and ketones), carboxylic acids and their derivatives, (e.g. acyl chlorides, esters), succinimidyl esters, isothiocyanates, sulfonyl chlorides, etc.

### Covalent and Non-Covalent Linking of Active Agents to Glycogen Nanoparticles

In one embodiment, there is provided an immunomodulator comprising glycogen nanoparticles linked to at least one molecule that induces or enhances an immune response in a subject. In one embodiment, the molecule that induces or enhances the immune response is covalently linked to the glycogen nanoparticles. In another embodiment, the glycogen nanoparticles are cationized and the molecule that induces or enhances the immune response is linked to the cationized glycogen nanoparticles via non-covalent interactions, in one embodiment ionic bonding.

A chemical compound bearing a functional group capable of binding to carbonyl-, cyanate-, imidocarbonate or amino-groups can be directly attached to functionalized glycogen nanoparticles. However, for some applications chemical compounds may be attached via a polymer spacer or a “linker”. These can be homo- or hetero-bifunctional linkers bearing functional groups such as amino, carbonyl, sulfhydryl, succimidyl, maleimidyl, and isocyanate, (e.g. diaminohexane), ethylene glycobis(sulfosuccimidylsuccinate), di sulfosuccimidyl tartarate, dithiobis(sulfosuccimidylpropionate), aminoethanethiol, etc.

In one embodiment, the modified glycogen nanoparticles are linked via covalent bond, directly or via a spacer, to a TLR agonist.

The nanoparticles may also be covalently linked directly or via a spacer, to one or more compounds such as biomolecules, small molecules, therapeutic agents, pharmaceutically active moieties, macromolecules, diagnostic labels, chelating agents, dispersants, surfactants, charge modifying agents, viscosity modifying agents, coagulation agents and flocculants, as well as various combinations of the above.

In one embodiment, the glycogen nanoparticles are covalently linked, directly or via a spacer, to a directing group for targeting to a specific cell type and/or cell compartment. For example, the nanoparticle could be covalently linked to an aptamer, small molecule, receptor ligand, growth factor, antibody or antibody fragment that would target the nanoparticle to a specific tissue or cell type.

Pharmaceutically useful moieties used as modifiers include hydrophobicity modifiers, pharmacokinetic modifiers, biologically active modifiers and detectable modifiers.

Chemical compounds covalently linked to glycogen nanoparticles may have light absorbing, light emitting, fluorescent, luminescent, Raman scattering, fluorescence resonant energy transfer, and electroluminescence properties.

For example, and without limiting the generality of the foregoing, an immunomodulator may comprise glycogen nanoparticles covalently bound to at least one molecule that induces or enhances an immune response (a TLR agonist) in a subject and the glycogen nanoparticles may further be covalently linked to a diagnostic or targeting label. In another example, cationized glycogen nanoparticles are non-covalently linked with a molecule that induces or enhances the immune response (a TLR agonist) and the nanoparticles are further covalently linked to a diagnostic or targeting label.

### Active Agents

In one embodiment nanoparticles as described herein are covalently or non-covalently linked to an agent that induces an immune response. In one embodiment, this agent is a TLR agonist.

In one embodiment the TLR agonist is a macromolecule, for example, a nucleic acid, a peptide, a peptidoglycan or a lipopolysaccharide.

In one embodiment, the size of the nucleic acid is not particularly restricted. The nanoparticle compositions described herein are suitable for the transport of nucleic acids having ≥10,000 base pairs. In one embodiment, the nucleic acid is between 10 and 10,000 nucleotides in length. In one embodiment, between 1000 and 10,000 nucleotides in length.

The TLR agonist may suitably be a double-stranded (ds) RNA, dsDNA or single-stranded (ss) RNA, ssDNA, or a synthetic analog of any of the foregoing (e.g. poly IC, CpG DNA, CpG ODNs, LNAs).

In one embodiment, the TLR agonist is an inducer of type 1 interferons (IFN) and the innate immune response.

In one embodiment, the TLR agonist is a synthetic (ds)RNA; in one embodiment polyinosinic: polycytidilic acid (PolyIC).

In one embodiment, the TLR agonist is CpG oligodeoxynucleotide (CpG ODN).

In one embodiment, the TLR agonist is a small molecule. In one embodiment the small molecule TLR agonist is a low molecular weight synthetic molecule of the imidazoquinoline family, including but not limited to imiquimod, resiquimod and vesatolimod.

In one embodiment, the TLR agonist may be selected from Table 1.

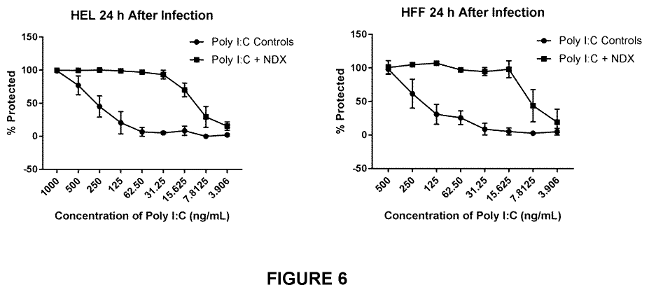

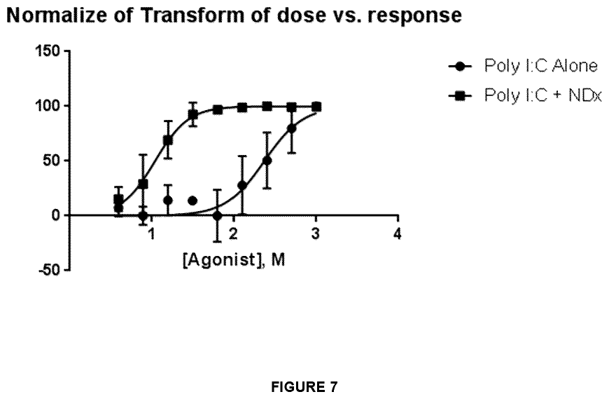

The present inventors have demonstrated that being bound to glycogen nanoparticles (whether covalently or non-covalently linked to the nanoparticles) as exemplified herein increases the stability of delivered TLR agonist within the cell. This contributes to enhanced signaling events, leading to a more robust and prolonged innate immune response (FIGS. 6-7).

As demonstrated in the Examples, the present inventors have found that immunomodulators as provided herein can be accumulated intracellularly by different types of cells. The nanoparticles can be further modified with specific tissue targeting molecules, such as folic acid, antibodies, aptamers, proteins, lipoproteins, hormones, charged molecules, polysaccharides, and low-molecular-weight ligands.

The TLR agonists, covalently or non-covalently linked to glycogen nanoparticles, as described herein provide a surprising and unexpected magnitude of enhancement of the innate immune response. As shown in the Examples, the innate immune activating potency of the exemplified TLR agonists is increased over 10-fold compared to unbound agonist. Without wishing to be bound by a theory, this is believed due to one or more of clustered presentation of TLR agonists to receptors, longer retention in intracellular compartments, and improved intracellular stability, resulting in enhanced signaling from Toll like receptors.

The combination of properties of glycogen nanoparticles described above together with the feasibility of low production costs, makes the nanoparticle compositions described herein highly suitable for the uses as described herein.

As demonstrated in the Examples, glycogen nanoparticles can carry molecules that enhance an innate immune response across the cell membrane, such molecules being effective within the cells to enhance an innate immune response.

In one embodiment, there is provided a method of treating or preventing a disease or condition in a subject comprising administering a therapeutically effective amount of an immunomodulator, comprising a glycogen-based nanoparticle, wherein the glycogen-based nanoparticle is covalently or non-covalently linked to a molecule that enhances an innate immune response.

Conditions that can benefit from enhanced innate immune response include, prophylactic and therapeutic treatment of viral diseases, therapeutic treatment of diseases resulting in immune suppression/dysfunction, and treatment of tumours/cancer alone or in combination with other therapies.

Aside from being used alone, immunomodulators as described herein may be used in combination with other therapeutics such as antivirals or anticancer agents, vaccines or other immunomodulators.

For example, glycogen nanoparticles administered intravenously in combination with another pharmaceutical, may boost the immune system thereby amplifying the action of the second pharmaceutical. In this case, the concentration of the other pharmaceutical could be potentially reduced, lowering toxicity.

### Formulation and Administration

Each glycogen particle is a single molecule, made of highly-branched glucose homopolymer characterized by very high molecular weight (up to 10⁷ Da). This homopolymer consists of α-D-glucose chains with 1→4 linkage and branching points occurring at 1→6 and with branching degree about 10%. These particles are spherical and can be manufactured with different sizes, in the range of 30 to 150 nm in diameter by varying the starting material and filtering steps. Glycogen nanoparticles are water soluble and by attaching compounds to glycogen nanoparticles they can be made water soluble. The high density of surface groups on the glycogen particles results in a variety of unique properties of glycogen nanoparticles, such as fast dissolution in water, low viscosity and shear thinning effects for aqueous solutions at high concentrations of glycogen nanoparticles. This is in contrast to high viscosity and poor solubility of linear and low-branched polysaccharides of comparable molecular weight. Furthermore, it allows formulation of highly concentrated (up to 30%) stable dispersions in water or DMSO.

As a function of the high amount of nucleic acid that can be transported, as well as the enhanced immune response associated with transporting, a biologically relevant induction of the innate immune response can be achieved at a much lower dose when non-covalently linked to PhG. For example, in an antiviral assay a dose of 62 ng/ml of poly IC:PhG was equivalent to 1000 ug/ml of none bound or “free” poly IC. For example, doses of free poly IC of 20 ug was sufficient to protect mice from viral infection. When formulated according to the present invention much lower doses would be effective (1.25 ug—calculated based on improvement over “free” agonist dosing provided by Zhao et. al, 2012).

The novel formulations of the invention may in some embodiments be admixed, encapsulated, or otherwise associated with other molecules, molecule structures or mixtures of compounds and may be combined with any pharmaceutically acceptable carrier or excipient. As used herein, a “pharmaceutically carrier” or “excipient” can be a pharmaceutically acceptable solvent, suspending agent or any other pharmacologically inert vehicle for delivering functionalized glycogen nanoparticles, whether alone or covalently linked to a biologically active or diagnostically useful molecule, to an animal.

The excipient may be liquid or solid and is selected, with the planned manner of administration in mind, so as to provide for the desired bulk, consistency, etc., when combined with glycogen nanoparticles and the other components of a given pharmaceutical composition. Examples of pharmaceutically acceptable carriers include one or more of water, saline, phosphate buffered saline, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the pharmacological agent.

In an embodiment, the compositions may be lyophilized or spray dried and may be subsequently formulated for administration. For example, in the case of a composition of a nucleic acid TLR agonist non-covalently linked to glycogen nanoparticles, the composition can be lyophilized or spray dried, yielding a product that is stable under storage/transport conditions that would not require cold chain (i.e. no need for refrigeration).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

For the purposes of formulating pharmaceutical compositions, monodisperse glycogen nanoparticles prepared as taught herein, may be provided in a dried particulate/powder form or may be dissolved e.g. in an aqueous solution. Where a low viscosity is desired, the glycogen nanoparticles may suitably be used in formulations in a concentration of up to about 25% w/w. In applications where a high viscosity is desirable, the nanoparticles may be used in formulations in concentrations above about 25% w/w. In applications where a gel or semi-solid is desirable, concentrations up to about 35% w/w can be used, or the nanoparticles can be used in combination with viscosity builders or gelling agent.

The pharmaceutical compositions of the present invention may be administered in a number of ways depending upon whether local or systemic treatment is desired and upon the area to be treated.

Without limiting the generality of the foregoing, the route of administration may be topical, e.g. administration to the skin or by inhalation or in the form of ophthalmic or optic compositions; enteral, such as orally (including, although not limited to in the form of tablets, capsules or drops) or in the form of a suppository; or parenteral, including e.g. subcutaneous, intravenous, intra-arterial or intra-muscular.

In another embodiment, the pharmaceutical compositions of the present invention are in the form of an implant. Suitably, these implants may be biocompatible, meaning that they will have no significant adverse effects on cells, tissue or in vivo function. Suitably, these implants may be bioresorbable or biodegradable (in whole or in part). Examples of implants include, without being limited to, tissue engineering scaffolds.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

The embodiments of the invention described above are intended to be exemplary only. The scope of the invention is therefore intended to be limited solely by the scope of the appended claims.

### EXAMPLES

### Example 1. Manufacture of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to cross flow filtration (CFF) using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

According to dynamic light scattering (DLS) measurements, the phytoglycogen nanoparticles produced had particle size diameter of 83.0 nm and a polydispersity index of 0.081.

### Example 2. Primary O-Aminoalkylation of Phytoglycogen with Aminoalkyl Halides or Hydrogensulfates

Phytoglycogen nanoparticles (PhG) are suspended in DMSO (10 mL/g PhG) and fully dissolved by stirring overnight at room temperature or at 50-80° C. for 1-4 hours. Sodium hydroxide powder (1.25 g/g PhG) is added slowly to the mixture and stirred another 1-2 hours. 2-bromoethylamine hydrobromide (1.63 g/g PhG), 2-chloroethylamine hydrochloride (1.21 g/g PhG) or 2-ethylamine hydrogensulfate (1.12 g/g PhG) in DMSO (7.5 mL/g PhG) is introduced to the PhG mixture in a single addition or dropwise over the course of 30 minutes, which is then left to stir at 25-90° C. for 4-16 hours (Table 2). The reaction mixture is neutralized with 0.1-0.5 M hydrochloric acid and the product is isolated by precipitation in ethanol (30 mL/g PhG) and centrifuged at 12,000×g for 10 minutes to pellet the product. The solids are re-suspended and washed twice more with equal aliquots of ethanol and pelleted, dried at 40° C. for 2-3 days or directly resuspended in water (20 mL/g PhG), and further purified by dialysis. Freeze-drying affords the product as a white solid. Yield: 93%; DS (conductometric titration): 0.002-0.003; diameter (DLS): 63 nm; PdI: 0.09; ZP: +2.3 mV.

TABLE 2
Reaction conditions and degrees of aminioalkylation (DS) for primary O-
aminoalkylated PhG.

| | Rate of aminating | Reaction | |
|---|---|---|---|
| Aminating agent | agent addition | temperature (° C.) | DS |
| 2-bromoethylamine | All at once | 25 | <0.01 |
| hydrobromide | | 40 | <0.01 |
| | | 90 | 0.02 |
| | Dropwise | 25 | 0.1-0.2 |
| | (0.2 mL/min) | | |
| 2- | All at once | 25 | 0.01 |
| chloromoethylamine | | | |
| hydrochloride | | | |
| Ethylamine | All at once | 25 | 0.005 |
| hydrogensulfate | | | |

### Example 3. Size Reductions of Phytoglycogen by Sulfuric Acid Hydrolysis and Subsequent O-Aminoethylation with 2-Bromoethylamine Hydrobromide

Phytoglycogen nanoparticles (PhG) are suspended in pH 2.0-3.4 sulfuric acid solution (39.4 mL/g PhG) and homogenized for 2 minutes. The solution is capped and stirred at 80-90° C. in a water bath. After 2-24 hours, the product is cooled in an ice-water bath and filtered through a 300 K membrane. After 5 cycles of 3-fold concentration at 30 psi, the solution is neutralized with 2M sodium hydroxide and lyophilized or spray dried to afford a white powder, which was subsequently aminated following methods of Example 2.

White powder; Yield: 58% from original PhG sample; diameter (DLS): 44.8 nm; PdI: 0.196; ZP: +4.3 mV.

### Example 4. Tertiary Aminoethylation of Phytoglycogen

Phytoglycogen nanoparticles (PhG) are dissolved in water (10 mL/g PhG) with stirring. Sodium hydroxide powder (0.63 g/g PhG) is added slowly and stirred for 20 minutes. 2-bromo-N,N-diethylethylamine hydrobromide (1.05 g/g PhG) in water (10 mL/g PhG) is introduced to the PhG solution, which is left to stir at room temperature overnight. The reaction mixture is neutralized with 0.1 M hydrochloric acid and dialyzed against deionized water for 6 cycles (12-14 kDa cut off). Freeze-drying affords the diethylaminoethylated (DEAF) product as a white solid. Yield: 83%; DS (NMR, conductometric titration): 0.36; diameter (DLS): 70 nm; PdI: 0.20; ZP: +45 mV.

### Example 5. Quaternary Ammonium Cationization of Phytoglycogen

Phytoglycogen nanoparticles (PhG) are mixed with aqueous sodium hydroxide solution (1.5-60.0 mmol of NaOH dissolved in 1-5 ml of water/g PhG) and heated to 25 or 45° C. Over the course of 10-120 minutes, 2,3-epoxypropyltrimethylammonium chloride in water (69% solution, 3.07 mL/g PhG) is added. Alternatively, 8.23 mmol of (3-chloro-2-hydroxy-prop-1-yl)dimethylalkyl ammonium chloride (alkyl=lauryl, cocoalkyl, stearyl) is stirred with 0.82 mL of 50% NaOH at 45° C. for 5 minutes, and 3.33 ml of 20% aqueous solution of PhG is added. Each reaction is stirred for another 2-6 hours at 45° C. Water (5-9 mL/g PhG) is then added, the mixture is cooled to room temperature and neutralized with 1 M HCl. The product is precipitated and washed in ethanol or hexanes (50-80 mL/g PhG), re-dissolved in water (20 mL/g PhG) and/or saturated NaCl (10 mL) and further purified by dialysis. Freeze-drying affords the product as a white solid. DS (NMR): 0.14-1.46 (Table 3).

### Example 6. Alkylation, Benzylation, or Silylation of Trimethylammonium-Cationized Phytoglycogen

Trimethylammonium-cationized PhG from Example 5 is oven-dried at 105° C. for 16 h (silylation) or used as is (alkylation/benzylation). The PhG was dissolved in dry dimethylsulfoxide (20 mL/g PhG) at 80° C. for 1 hour. For alkyl/benzylation reactions, water (0.5 mL) and 50% NaOH (0.042-2.47 mmol/g PhG) is added and stirred vigorously for 10 minutes. Alkyl or benzyl halides (0.51-30.6 mmol/g glycogen) are then added and the mixture is stirred for 2 hours at 60° C., cooled to room temperature and neutralized with glacial acetic acid. For benzylation, the addition of benzyl bromide (0.51 mmol/g PhG) could be performed at 60° C. for 2 hours directly following cationization at 45° C. for 2-6 hours, as a one-pot synthesis. For silylation, the reaction vessel is capped with a rubber septum, cooled to 0° C., and triethylamine (1.19-4.75 mmol/g PhG) is added, followed by dropwise addition of silyl chloride (trimethylsilyl chloride (0.36-1.46 mmol/g PhG), triethylsilyl chloride (0.36 mmol/g PhG)) and stirred overnight at room temperature. For alkylation/benzylation, the crude mixture is extracted several times with diethyl ether/hexanes/ethanol and re-suspended in saturated aqueous NaCl of pH 5-7, then dialysed and freeze-dried; whereas for silylation, the product is precipitated into acetonitrile and washed with hot acetonitrile, then dried overnight at 60° C. and 100 mbar. All are white solids. DS(NMR): alkylation 0.004-1.58; one-pot benzylation: 0.068 for benzyl group and 1.12 for cationic group; silylation 0.19-0.45 (Table 3).

TABLE 3
Reaction parameters and characterization data for
Example 5 and Example 6.

| | | | Cationi- | | | |
|---|---|---|---|---|---|---|
| | | Alkyl | zing | | Hydro- | ζ- |
| | NaOH | halide | agent | | dynamic | poten- |
| | (mmol/ | (mmol/ | (mmol/ | | diameter | tial |
| Substituent | g PhG) | g PhG) | g PhG) | DS_{NMR} | (DLS) | (mV) |
| Ethyl | 0.04 | 0.51 | — | 0.006 | 61.95 | 53.3 |
| Ethyl | 0.17 | 2.04 | — | 0.083 | 64.81 | 51.4 |
| Ethyl | 0.41 | 5.10 | — | 0.66 | 64.38 | 51.3 |
| Dodecyl | 0.04 | 0.51 | — | 0.004 | 76.91 | 57.1 |
| Dodecyl | 0.17 | 2.04 | — | 0.17 | 166 | 33.3 |
| Benzyl | 0.04 | 0.51 | — | 0.052 | 64.57 | 57.9 |
| Benzyl | 0.17 | 2.04 | — | 0.528 | 70.19 | 57.5 |
| Benzyl | 0.41 | 5.10 | — | 1.2 | 78.64 | 62.5 |
| Benzyl | 0.99 | 12.24 | — | 1.58 | 65.61 | 51.6 |
| Benzyl/ | 2.5 | 0.51 | 12.4 | 0.068 | 49.98 | 39.9 |
| QUAB 151 | | | | | | |
| QUAB 151 | 2.35 | — | 0.28 | 0.34 | 63.81 | 37.4 |
| QUAB 151 | 2.35 | — | 0.56 | 0.47 | 59.18 | 37.6 |
| QUAB 342 | 15.5 | — | 12.5 | 0.88 | 62.74 | 67.7 |
| QUAB 360 | 15.5 | — | 12.5 | 1.02 | 76.58 | 52.6 |
| QUAB 426 | 15.5 | — | 12.5 | 0.136 | 165.7 | 57.7 |

Abbreviations:
QUAB, (3-chloro-2-hydroxy-prop-1-yl)dimethylalkyl ammonium chloride (alkyl = lauryl (342), cocoalkyl (360), stearyl (426)), QUAB 151: (3-chloro-2-hydroxy-prop-1-yl)trimethylammonium chloride.

### Example 7. Etherification of Phytoglycogen with N-Protected Aminoalkyl Halides or Epoxides

Phytoglycogen nanoparticles (PhG) are dissolved in water or DMSO (20 mL/g PhG) with stirring. In the case of DMSO solvent, the solution is heated to 80° C. for 1 hour to dissolve the PhG. Sodium hydroxide powder (1.0 g/g PhG) or a 1.1 M solution of dimsyl lithium in DMSO (for the DMSO reaction, 40 mL/g PhG) is added slowly and stirred for 10-90 minutes. To the solution at room temperature, phthalimide (N-(2-bromoethyl), 2.4 g/g PhG; or N-(2,3-epoxypropyl), 1.9 g/g PhG) 1.05 g/g PhG) was added as a solid (along with optional tetrabutylammonium bromide; 6 g/g PhG) and the suspensions are stirred at 20-45° C. for 1-2 days. The yellow reaction mixture is neutralized with 0.1-0.5 M hydrochloric acid, rinsed with ethanol, and dialyzed against deionized water for 6 cycles (12-14 kDa cut off). Freeze-drying affords the product as a white to off-white solid. Yield: 0.27-1.27 g; DS (NMR): 0.02-0.33; diameter (DLS): 62-71 nm; PdI: 0.11-0.20; ZP: −13-−45 mV.

### Example 8. Deprotection of Imides to Produce O-Aminoalkyl Phytoglycogen

N-protected derivative of PhG from Example 7 (50-100 mg) is dissolved in water (3.5 mL) and NaBH4 (59.2 mg) is added. After stirring 17 hours, glacial acetic acid (0.4 mL) is carefully added to bring the solution pH to 5. Once the foaming subsides, the vial is stoppered and heated to 80° C. with stirring for 2 hours. Alternatively, the PhG is dissolved in aqueous hydrazine hydrate (3.5 mL), stirred at room temperature for 2 hours, 2% hydrochloric acid (3.5 mL, to reach pH 8) is added, and the mixture is stirred at room temperature for 16 hours. A third variation involves suspending PhG in 40% aqueous methylamine (2.1 mL), refluxing 4 hours (˜48° C.), then diluting with an equal amount of water and acidifying (pH<2) with 0.1 M hydrochloric acid, and ultimately readjusted to pH-5. Room temperature mixtures are dialyzed against deionized water for 6 cycles (12-14 kDa cut off). Freeze-drying affords the product as a white solid. Diameter (DLS): 52.6 nm; PdI: 0.23; ZP: −33.4 mV.

### Example 9. Cyanoalkylation of Phytoglycogen with Cyanoalkenes and Cyanoalkyl Halides

Phytoglycogen nanoparticles (PhG) are pre-treated with a 15% w/v solution of TMAC in water (2-3 mL) or used as is. The PhG mixture is slurried in acrylonitrile (24 mL/g PhG), and 12% NaOH (1 mL/g PhG) is added. The slurries are stirred at room temperature for 5 h. The mixture becomes doughy. The mixtures are washed with ethanol (30 mL/g PhG) and the slurry is poured into water (20 mL/g PhG) which is heated to evaporate off excess acrylonitrile in vacuo. The product is filtered and dried at 40° C. under vacuum overnight. diameter (DLS): 84-101 nm; PdI: 0.17-0.23; ZP: −23-−24 mV.

### Example 10. Reduction of Cyanoalkyl Phytoglycogen

The reaction vessel is oven-dried overnight. THF is distilled to remove stabilizers and dried over molecular sieves prior to use. Cyanoalkyl PhG (400 mg) is dispersed in 6 mL of dry THF in an oven-dried 100 mL three-neck RBF equipped with a condenser, a stir bar, and three septum plugs. The system is flushed with nitrogen. 2 mL of 1M borane-THF is added dropwise by syringe to the reaction and left to reflux under an atmosphere of dry nitrogen (balloon, occasional replacement as needed) for 3-5 h. 3M methanolic HCl is added slowly (prepared from 3.8 mL of trimethylsilyl chloride and 6.2 mL of chilled methanol). The mixture is cooled to 0° C., and methanolic HCl is added dropwise (˜8 mL, or until the pH reaches <2). The reaction is brought to reflux with stirring for another hour. The reaction is cooled, solvents are evaporated in vacuo and the residue is treated with an additional 10 mL of MeOH. Once the volatile components have been removed by evaporation, the product is dispersed in deionized water (10-20 mL) and the pH is adjusted to 11 with 50% NaOH solution. The solution at pH 11 is dialyzed (12-14 kDa cut-off) against RO water for 6 cycles, or until solution pH is constant at 8-9. The product is then (frozen and) lyophilized to dryness.

### Example 11. Hydroxypropylation of Phytoglycogen

Phytoglycogen nanoparticles (PhG) are dissolved in water (4-10 mL/g PhG) and homogenized. Powdered sodium hydroxide (0.8 g/g PhG) is added and stirred to dissolve in the mixture. Over the course of 30 minutes, propylene oxide (0.5-4.0 mL/g PhG) is added dropwise to the PhG solution and agitated to combine two immiscible phases. The reaction is stirred for 16-20 hours. The mixtures are neutralized with 0.5 M hydrochloric acid, dialyzed (12-14 kDa cut-off) against RO water for 6 cycles, and lyophilized to afford a white powder.

### Example 12. TEMPO Oxidized Phytoglycogen

Phytoglycogen nanoparticles (PhG) are dissolved in glycine buffer (0.05 M, pH 10.20, 33 mL/g PhG). The sample is placed in an ice bath. After 4 hours, a solution of TEMPO (0.04 g/g PhG) in glycine buffer (1.7 mL/g PhG) is added to the reaction mixture. NaBr (0.60 g/g PhG) is then added. After an hour (reaction at 3° C.), NaClO solution (4.52% chlorine) is introduced over the course of 30 minutes (80 mL/g PhG per addition). The reaction mixture is stirred at 0-5° C. for 72 hours, then quenched with anhydrous ethanol (13 mL/g PhG). The mixture is dialyzed (12-14 kDa cut-off) against RO water for 6 cycles, and lyophilized to afford an off-white powder.

### Example 13. Poly IC Bound Phytoglycogen

All solutions and glassware were sterile or autoclaved (121° C. for 30 minutes) and nuclease-free where possible. 1.3 mg of native PhG (prepared as in Example 1) or TEMPO-oxidized PhG (Example 12) is dissolved in 0.5 M MES solution, pH 6.4 (0.5 mL). In a separate vial, poly IC (polyinosinic:polycytidylic acid, 4.2 mg) is dissolved in PBS solution (0.5 mL). The PhG and poly IC solutions were stirred at 50° C. for 30 minutes. In a third vial, MES solution at room temperature (3.4 mL) was set aside and 0.1 mL of neat EDC (N-(3-dimethylaminopropyl)-N′-ethylcarbodiimide) was added, and the pH was adjusted to 6.7 with NaOH. 1 mL of this EDC/MES solution was added to poly IC-PBS solution, which was transferred into PhG-MES solution. Two controls were also prepared: one without Poly IC, and one without EDC. All reactions were left to stir at 50° C. for 1 hour, then cooled to room temperature. Each reaction mixture was transferred into 100K centrifugal filtration devices (3×0.5 mL) and spun down at 15,000×g for 10 minutes to recover the retentate. Alternatively, samples were dialyzed (MWCO 12-15,000 Da against RO water) for 2 days and lyophilized, or precipitated in ethanol (20 mL) then pelleted by centrifugation (15 min at 7500×g) and dried at 25-50° C. for 6-24 hours.

### Example 14. Cytotoxicity of Glycogen/Phytoglycogen in Cell Cultures

The effects of the glycogen/phytoglycogen nanoparticles on cell viability was analyzed to assess cytotoxicity of the particles. Glycogen/Phytoglycogen nanoparticles were extracted from rabbit liver, mussels, and sweet corn using cold-water and isolated as described in Example 1.

Cationic amphiphilic particles were prepared by functionalizing phytoglycogen with amino-groups, quaternary ammonium groups and alkyl chains of various chain length as described in Example 2 and Example 6.

Cell lines used: rainbow trout gill epithelium (RTG-2).

To measure changes in cell viability two fluorescence indicator dyes were used, alamar blue (ThermoFisher) and CFDA-AM (Thermofisher); these dyes measure cell metabolism and membrane integrity respectively. For these dyes, more fluorescence indicates more viable cells.

None of the assays detected any cytotoxicity effects in cells after 48 h incubation in the presence of glycogen or phytoglycogen or their derivatives at concentrations of 0.1-10 mg/ml.

### Example 15. Monodisperse Phytoglycogen Nanoparticles as a Carrier for Double-Stranded RNA

RTgutGC, a rainbow trout gut origin cell line, obtained from N. Bols (University of Waterloo) were used in this study. RTgutGC was routinely cultured at 20 degrees Celsius in 75 cm² plastic tissue culture flasks (BD Falcon, Bedford, Mass.) with Leibovitz's L-15 media supplemented with 10% fetal bovine serum (FBS) and 1% penicillin/streptomycin (P/S).

Poly IC was stored in stock solutions of 10 mg/mL diluted in PBS (Sigma-Aldrich, St Louis, Mo., USA) and stored at −20° C. Poly IC was covalently linked to PhG (Example 13) and made into aliquots of 10 mg/mL PhG covalently linked to 1.44 mg/mL of poly IC. The covalently linked PhG:poly IC was also stored at −20° C.

RTgutGC was seeded at 8×10⁵ cells/well in 6-well plates and left to grow for 24 hours. Cells were treated with media alone (control), 1 ug/mL of poly IC, 1 ug/mL of poly IC covalently linked to 6.875 ug/mL PhG (all using 1×L-15 supplemented with 10% FBS) and incubated at 20° C. for 24 hours. RNA was extracted using Trizol, following the manufacturer's instructions. cDNA synthesis using an iScript cDNA synthesis kit (Bio-Rad) was completed using 1 μg of RNA, 4 uL of iScript and up to 20 uL DNA quality water in each reaction. The cDNA was diluted in 1 in 10 in DNA quality water prior to qPCR reactions.

All PCR reactions contained: 2 uL of diluted cDNA, 2× SsoFast EvaGreen Supermix (Bio-Rad), 0.2 um forward primer, 0.2 μM reverse primer and nuclease-free water to a total volume of 104, (the housekeeping gene actin primers were at 0.1 μM). The qPCR program was 98° C. 2 mins, 40 Cycles of 98° C. 5 s, 55° C. 10 s and 95° C. for 10 s. A melting curve was completed from 65° C. to 95° C. with a read every 5 s. Gene expression was normalized to the housekeeping gene (β actin) and expressed as a fold change over the untreated control group.

TABLE 4
Primers designed for qPCR experiments

| Target | Forward primer (5′-3′) | Reverse Primer (5′-3′) |
|---|---|---|
| Human isg15 | cagccatgggctgggac | cttcagctctgacaccgaca |
| Human interferon beta | aaactcatgagcagtctgca | aggagatcttcagtttcggagg |
| Human cxc110 | gaaagcagttagcaaggaaagg | gacatatactccatgtagggaagtg |
| Human ifit2 | gcctaatttacagcaaccatga | tcatcaatggataactcccatgt |
| Human beta actin | ctggcacccagcacaatg | ccgatccacacggagtacttg |
| Rainbow trout actin | gtcaccaactgggacgacat | gtacatggcaggggtgttga |
| Rainbow trout vig3 | atggaaaggcagaggctgtc | tgagtgggttctgtaatcagca |

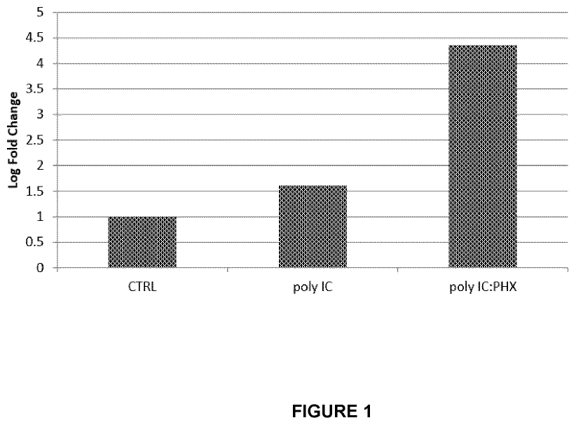

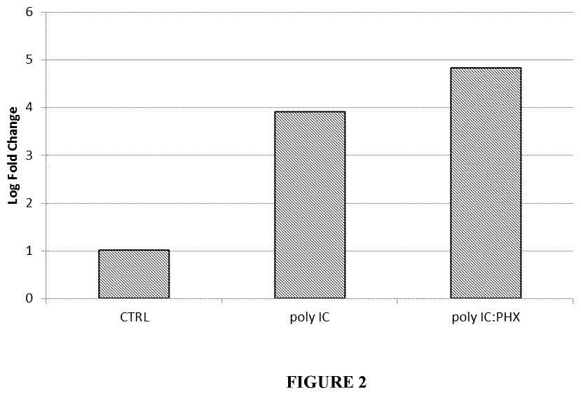

Referring to FIGS. 1 and 2, the results provide evidence that poly IC:PhG induces a stronger antiviral response compared to poly IC alone. This is based on the qRT-PCR data cited, which shows that both IFN1 and vig3 (an ISG) expression levels were higher in the poly IC:phytoglycogen nanoparticle treated cells compared to poly IC alone. This demonstrates that phytoglycogen nanoparticles can be used as a carrier for nucleic acid-based therapeutics that require the nucleic acid to enter the cell.

### Example 16. Binding of Poly IC by Cationic PhG

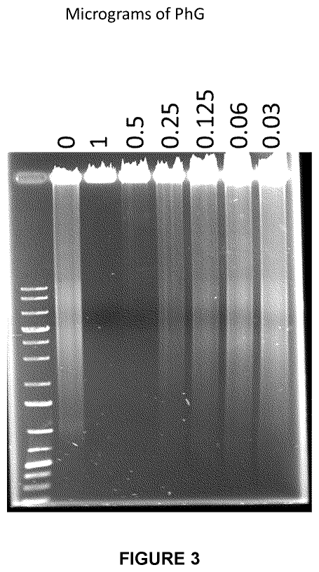

The ability of cationic phytoglycogen nanoparticles (prepared according to Example 5 table 3) (Cat-PhG) to bind poly IC was demonstrated by electrophoretic mobility shift assay (EMSA). Three (3) micrograms of poly IC was mixed with 2-fold dilution series of Cat-PhG with DS 0.88 (Example 5). After a 20-minute incubation at room temperature, samples were separated on a 1% agarose gel, stained with ethidium bromide, and subsequently imaged under UV light. Shown in FIG. 3 are the EMSA gels for Cat-PhG-0.88 (Example 5) demonstrating maximum loading ˜6:1 (poly IC:PhG w/w).

### Example 17. PhG Bound TLR 3 Agonist Induces Greater Expression of Interferon Stimulated Genes than Free Agonist

PhG bound TLR 3 agonists induce greater expression of interferon stimulated genes than “free” agonist. Quantitative reverse-transcriptase polymerase chain reaction (qRT-PCR) reactions were used to quantify the amount of ISG transcript in mock, “free” poly IC treated, or PhG-poly IC treated Human Embryonic Lung (HEL-299) and Human Foreskin Fibroblast (HFF-1) cells. A) HEL-299 or B) HFF-1 cells were plated in 6-well tissue culture dishes at approx. 80% confluency. The next day cells were mock treated, or treated with 1 ug/ml of poly IC, or 1 ug/ml poly IC coupled to cationic PhG at a 1:1 ratio (prepared according to Example 16). After a 6-hour incubation at 37° C., total RNA was isolated via Trizol reagent (Thermo-Fisher). Subsequently, 500 ng of total RNA was converted to cDNA using iScript (BioRad); cDNA was diluted 1:10 and 2 μl was used in a qPCR reaction (EvaGreen, BioRad) with primers specific for human actin, IFN-beta, IFIT, ISG15 and CXCL10. All transcripts were normalized to actin and quantified relative to mock treated. Cells treated with Cat-PhG-poly IC demonstrated greater transcript levels of all ISGs assayed compared to both mock and “free” poly IC treated cells in both HEL-299 and HFF-1 cells.

### Example 18. Protection from Viral Infection by PhG Bound TLR3 Agonist and Free Agonist

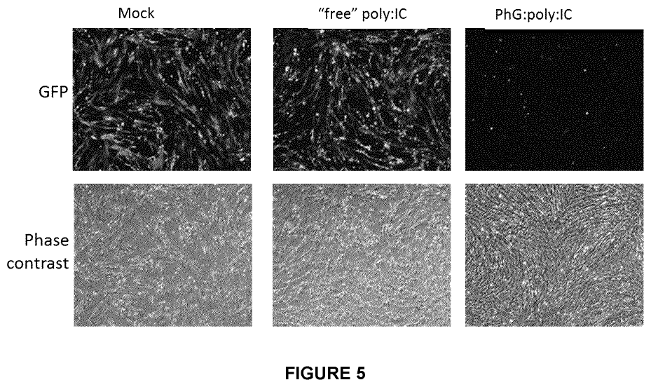

HEL-299 or HFF-1 cells were plated in a 6-well or 12-well dish at 80% confluency. The next day cells were treated with a 2-fold dilution series (1000-3.906 ng/ml) of “free” poly:IC, or poly:IC coupled to cationic PhG at a 1:1 ratio (w/w) (prepared according to Example 16). After 1 or 6-hours of treatment cells were infected with vesicular stomatitis virus-green fluorescent protein (VSV-GFP) at a multiplicity of infection (MOI) of 0.1. FIG. 5 shows infection monitored by GFP fluorescence via fluorescent microscopy. FIG. 6 shows percent infection determined by measuring GFP fluorescence via plate reader. The PhG:poly IC demonstrated complete protection up to 31.25 ng/ml in HEL, compared to 1000 ng/ml of free poly IC. A similar trend was observed in HFF-1 cells with complete protection in the PhG:poly IC group at 15.625 ng/ml while the free poly IC group had complete protection at 500 ng/ml. FIG. 7 shows PhG bound TLR3 agonist reduced the EC50 of “free” agonist greater the 20-fold.

Example 14 through 18 demonstrate that innate immune stimulating molecules, when bound to PhG, can provide greater induction of innate immune genes than unbound material, and that the induction is sufficient to protect cells from viral infection.

### Example 19. Enhanced Uptake and Intracellular Stability of TLR 3 Agonist Non-Covalently Linked to PhG in Rainbow Trout Gut Cells (RT-Gut)

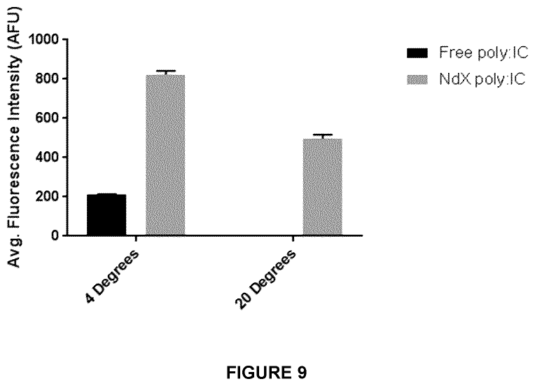

FIG. 9 shows PhG enhances uptake and intracellular stability of TLR 3 agonist in rainbow trout gut cells (RT-gut). Low molecular weight poly IC was labelled with the Ulysis AlexaFluor 546 labelling kit (Invitrogen), then left as “free” poly IC or coupled with cationic PhG (0.38) at a 1:1 (w/w) ratio. Free poly IC or PhG coupled poly IC was added to RT-gut cells at a concentration of 1 ug/ml and incubated at 4° C. or 20° C. for 4 hours. Cells were imaged via fluorescent microscopy, and average intracellular fluorescence intensity was measured from 5 randomly selected cells. PhG poly IC treated cells had a higher fluorescence than free poly IC; no fluorescence was detected from free poly IC treated cells for the 20 degree treatment.

### Example 20. Enhanced Uptake and Intracellular Stability of TLR 9 Agonist Non-Covalently Linked to PhG in Chicken Macrophage Cell Line HD-11 Cells

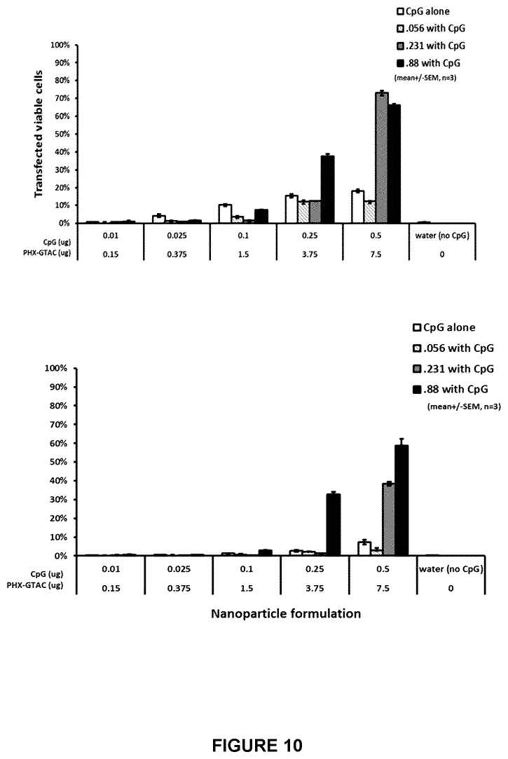

FIG. 10 shows PhG enhances uptake and intracellular stability of TLR 9 agonist in Chicken macrophage cell line HD-11 cells. HD-11 Cells, 30,000 per well, were seeded into an untreated 96-well U-bottom plate and suspended with 250 μL basic media. Cells in each well were dosed in triplicate with different doses of PhG-CpG formulations and incubated at 37° C. for 2 hours in basic media. After removing the cell supernatants from the treated cells, the cell pellets were re-suspended in 250 μL of PBS mixed with MitoTracker™ Green FM (Life Technologies) cell viability stain for flow cytometry. The CpG-ODN uptake and cell viability were assessed using the Attune® Acoustic Focusing Flow Cytometer (Applied Biosystems, Life Technologies, Carlsbad, Calif., USA). CpG uptake was measured as percentage of cells positive for Alexa Fluor 647 CpG-ODN red fluorescence following treatment. The viability was assessed using MitoTracker™ Green FM viability dye. The CpG-ODN uptake was calculated based on the percentage of viable cells that exhibited a fluorescence signal above the threshold signal.

Example 19 and 20 evidence that PhG bound material generates increased innate immune responses due to better uptake, retention, and intracellular stability of innate immune stimulators.

### Example 21. PhG Enhances Macrophage Stimulation by TLR9 Agonists at Low Concentrations

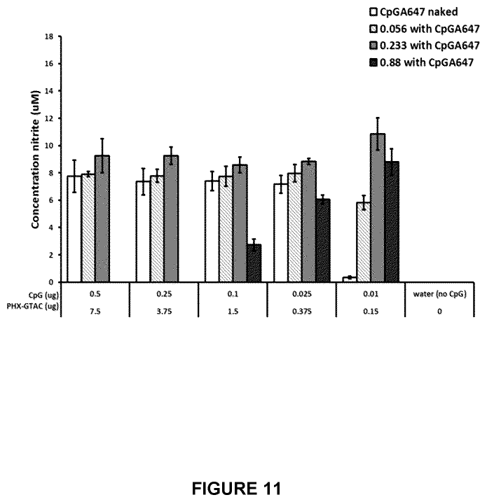

FIG. 11 shows PhG enhances macrophage stimulation by TLR9 agonists at low concentrations. HD-11 Cells, 30,000 per well, were seeded into an untreated 96-well U-bottom plate and suspended with 250 μL basic media. Cells in each well were dosed in triplicate with different doses of PhG-CpG formulations and incubated at 37° C. for 2 hours in basic media. After the 24-hour incubation, the Griess assay (standard Griess Assay Kit, Life Technologies) was used to evaluate nitrite production. Briefly, 150 μL of cell supernatants were transferred to a 96-well clear bottom plate pre-filled with 130 μL sterile water. The assay was carried out as per manufacturer's protocol. The absorbance was read at 548 nm using a microplate reader and nitrite concentration was calculated using a nitrite standard curve (1-100 μM). Statistical analysis was performed using the GraphPad Prism software. CpG bound PhG demonstrated higher nitrite production than “free” CpG at the 0.01 μg concentration.

Example 14 through 21 demonstrate that TLR agonists bound to PhG generate more robust induction of the innate immune system in both immune and non-immune cells, when compared to unbound agonist.

### Example 22. Internalization of Cy5.5-Labeled Glycogen/Phytoglycogen Particles by THP-1 Monocytes

Conjugation of a near-infrared fluorescent dye (Cy5.5) to the particles used in this study enabled analysis of nanoparticle uptake by confocal fluorescence microscopy. Cy5.5-labeled phytoglycogen particles were produced as described below.

100 mg of phytoglycogen nanoparticles, produced according to Example 1, was suspended in 20 mL of 0.1 M Sodium bicarbonate buffer, pH 8.4. With a temperature probe in a control vial (containing 0.1 M Sodium bicarbonate buffer), the reaction vessel containing the solution was wrapped in aluminium foil and placed on a hot plate at 35° C. 1 mg Cy5.5-NHS ester (Lumiprobe Corp.) was suspended in 4 mL DMF. During a 1 h period, Cy5.5-NHS ester was added in 1-mL aliquots. The pH of solution was constantly checked before and after addition, adjusting to 8.4 with the addition of a 2 M HCl solution. After the final aliquot of Cy5.5-NHS ester in DMF was added, the pH was monitored and adjusted as needed. The reaction was allowed to proceed for 2 h further, after which the pH was adjusted to 4.0 with a 2 M HCl solution as aforementioned.

To the acidified solution containing the resulting polysaccharide nanoparticle-Cy5.5 conjugate was added 2 volumes of ethanol. This solution was cooled to 4° C. and centrifuged at 6000 rpm for 15 minutes. After centrifugation, the supernatant was poured off and the pellet was resuspended in 15 mL deionized water. 2 volumes of ethanol was added to the resuspended pellet and it was cooled and centrifuged as before. This was repeated one time further until the supernatant that was poured off was clear and colourless. The pellet was resuspended a final time in 10 mL anhydrous Diethyl ether via use of a homogenizer. The resulting conjugate was rendered by evaporating to dryness with trace heat.

THP-1 cells were incubated with Cy5.5-labeled glycogen/phytoglycogen particles at a concentration of 1 mg/ml at 4° C. (negative control) and 37° C. for 0.5, 2, 6 and 24 h. Then cells were washed with PBS, fixed in 10% Buffered Formalin Solution and washed again with PBS. Than fixed cells were stained with DAPI (nucleus) and AF488 (cell membrane). Internalization of phytoglycogen particles was assessed by Olympus Fluoview FV1000 Laser Scanning Confocal Microscope.

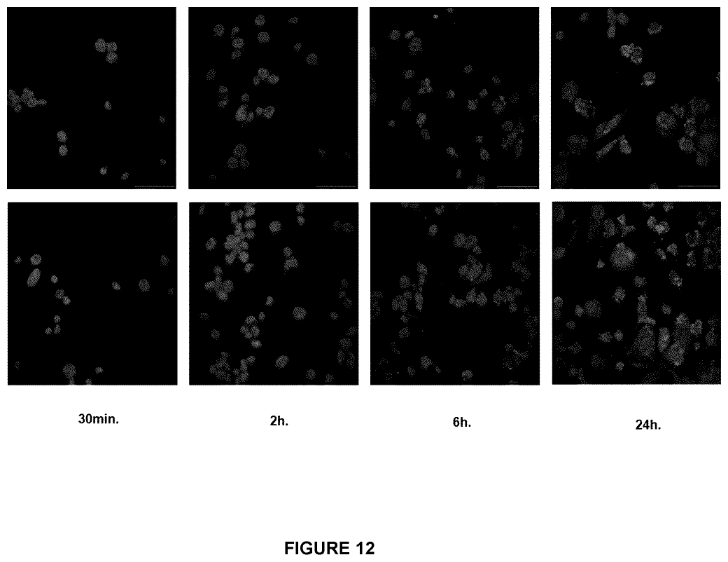

Incubation at 4° C. when endocytotic and phagocytotic processes are no longer active, did not result in any particles associated with THP-1 cells (FIG. 12). This confirmed, that there was no accumulation of the nanoparticles by THP-1 cells due to the surface binding. In contrast, incubation at 37° C. for over 6 hours revealed considerable accumulation of Cy5.5-labeled phytoglycogen particles in cell cytoplasm (FIG. 12). However, there was low uptake in the time interval of 0.5-2 h.

1. An immunomodulator for enhancing the innate immune response comprising: a TLR agonist covalently or non-covalently linked to glycogen-based polysaccharide nanoparticles having a molecular weight of 10⁶ to 10⁷ Da comprising α-D glucose chains, having an average chain length of 11-12, with 1→4 linkage and branching point occurring at 1→6 and with a branching degree of between 6% and 13%.

2. The immunomodulator of claim 1, wherein the TLR agonist is selected from: double-stranded RNA, double-stranded DNA and single-stranded RNA, single-stranded DNA, or any synthetic analogs thereof including poly IC, CpG ODN, LNA.

3. The immunomodulator of claim 1, wherein the TLR receptor is located in an endosome.

4. The immunomodulator of claim 1, wherein the TLR receptor is located at the cell surface.

5. The immunomodulator of claim 1 wherein the TLR agonist comprises between about 60% and 600% by weight relative to the polysaccharide nanoparticles.

6. The immunomodulator of claim 1, wherein the nanoparticles have a polydispersity index (PDI) of less than about 0.3 as measured by dynamic light scattering and an average particle diameter of between about 10 nm and 150 nm.

7. The immunomodulator of claim 1, wherein the nanoparticles are further covalently linked to one or more small molecules for directing the nanoparticles to a type of cell or cellular compartment.

8. The immunomodulator of claim 1 wherein the TLR agonist is covalently linked to the nanoparticles through a linking group.

9. The immunomodulator of claim 1 wherein the nanoparticles are cationic and the TLR agonist is non-covalently linked to the nanoparticles through electrostatic interactions.

10. A pharmaceutical composition comprising the immunomodulator of claim 1 and a pharmaceutically acceptable excipient.

11. The pharmaceutical composition of claim 10 wherein the composition is a powder, tablet or capsule.

12. The pharmaceutical composition of claim 10 wherein the composition is a vaccine.

13. The pharmaceutical composition of claim 10 further comprising an antiviral agent, an anticancer agent, a further immunomodulator, or a vaccine.

14. A method of stimulating an innate immune response in a subject comprising administering to the subject a therapeutically effective amount of an immunomodulator according to claim 1.

15. The method of claim 14, wherein the immunomodulator is administered by intramuscular, oral, sublingual, buccal, parenteral, nasal, subcutaneous, intradermal, or topical administration.

16. The method of claim 14 for preventing or treating a viral infection in the subject.

17. The method of claim 14 for cancer immunotherapy.

18. A method of potentiating a TLR agonist comprising covalently or non-covalently linking the TLR agonist to glycogen-based polysaccharide nanoparticles having a molecular weight of 10⁶ to 10⁷ Da comprising α-D glucose chains having an average chain length of 11-12, with 1→4 linkage and branching point occurring at 1→6 and with a branching degree of between 6% and 13%.

19. The method of claim 18, wherein the nanoparticles are cationized.

20. The method of claim 18 wherein the TLR agonist is selected from: double-stranded RNA, double-stranded DNA and single-stranded RNA, single-stranded DNA, or any synthetic analogs thereof including poly IC, CpG ODN, LNA.


SEQUENCE LISTING (14 sequences)

SEQ ID NO: 1 - DNA, 17 bp, Artificial Sequence
  primer sequence
cagccatgggctgggac

SEQ ID NO: 2 - DNA, 20 bp, Artificial Sequence
  primer sequence
cttcagctctgacaccgaca

SEQ ID NO: 3 - DNA, 20 bp, Artificial Sequence
  primer sequence
aaactcatgagcagtctgca

SEQ ID NO: 4 - DNA, 22 bp, Artificial Sequence
  primer sequence
aggagatcttcagtttcggagg

SEQ ID NO: 5 - DNA, 22 bp, Artificial Sequence
  primer sequence
gaaagcagttagcaaggaaagg

SEQ ID NO: 6 - DNA, 25 bp, Artificial Sequence
  primer sequence
gacatatactccatgtagggaagtg

SEQ ID NO: 7 - DNA, 22 bp, Artificial Sequence
  primer sequence
gcctaatttacagcaaccatga

SEQ ID NO: 8 - DNA, 23 bp, Artificial Sequence
  primer sequence
tcatcaatggataactcccatgt

SEQ ID NO: 9 - DNA, 18 bp, Artificial Sequence
  primer sequence
ctggcacccagcacaatg

SEQ ID NO: 10 - DNA, 21 bp, Artificial Sequence
  primer sequence
ccgatccacacggagtacttg

SEQ ID NO: 11 - DNA, 20 bp, Artificial Sequence
  primer sequence
gtcaccaactgggacgacat

SEQ ID NO: 12 - DNA, 20 bp, Artificial Sequence
  primer sequence
gtacatggcaggggtgttga

SEQ ID NO: 13 - DNA, 20 bp, Artificial Sequence
  primer sequence
atggaaaggcagaggctgtc

SEQ ID NO: 14 - DNA, 22 bp, Artificial Sequence
  primer sequence
tgagtgggttctgtaatcagca